(19) United States
(12) Patent Application Publication
(10) Pub. No.: US 2009/0132048 A1
(43) Pub. Date: May 21, 2009

(54) Biodegrading Coatings of Salt for Protecting Implants Against Organic Contaminants

(75) Inventor: Alain J. Denzer, Laufenburg (CH)

Correspondence Address: SHOEMAKER AND MATTARE, LTD, 10 POST OFFICE ROAD - SUITE 100, SILVER SPRING, MD, 20910, US

(73) Assignee: CAMLOG BIOTECHNOLOGIES AG, Basel (CH)

(21) Appl. No.: 12/293,125

(22) PCT Filed: Mar. 7, 2007
(86) PCT No.: PCT/EP07/52139

§ 371 Date: Sep. 16, 2008

(30) Foreign Application Priority Data
Apr. 13, 2006 (EP) 06112659.5

(51) Int. Cl. A61F 2/28; A61C 8/00; A61B 19/02; B05D 3/00

(52) U.S. Cl. 623/16.11; 433/201.1; 206/438; 427/2.26

(57) ABSTRACT

An implant, in particular an implant for dental applications, is provided at least partially in the area of its surface with a protective layer. The protective layer is intended to avoid the deposition of contaminants. The protective layer is chosen such that it breaks up on contact with body fluids and/or bone, with the result that essentially no residues remain on the surface of the implant.

This invention relates to an implant, a package comprising an implant and a process for treating an implant having the features of the preamble of the independent claims. Implants, including in particular dental implants, are being used in large volumes. Such implants are for insertion into bones, for example into the jawbone. Such implants preferably consist of titanium or of alloys based on titanium. An important property of an implant is its osteointegration time, i.e., the time until the implant is sufficiently firmly bonded to the surrounding bone substance.

The chemical state of the surface of titanium or titanium-based alloys is complex. It is known that the surface of titanium metal spontaneously oxidizes in air and water, and it is believed that a reaction with water takes place at the surface, i.e., in the outermost layer of atoms, to form titanium hydroxyl (TiOH) groups (Boehm H. P., 1971, Acidic and basic properties of hydroxylated metal oxide surfaces, Discussions Faraday Society, 52, 264-275).

Baier (1972, The role of surface energy in thrombogenesis, Bull. N.Y. Acad. Med. 48, 257-272) developed a model for the contact between blood and biomaterial, positing a correlation between bio-compatibility, bioadhesion and the surface tension of the solid body, or of the contact angle computed therefrom. According to that model, a hydrophilic surface of contact angle 0-31° possesses very strong bioadhesion. By contrast, contact angles in the range >70° correspond to hydrophobic surfaces and to a hypothetical zone of biocompatibility. The wetting properties or the hydrophilic character of the implant surface can be determined in a conventional manner by measuring the contact or wetting angle between the liquid (water) and the dry metallic substrate surface using optical methods. To determine the contact angle, the coated surface is washed with pure water and dried in pure nitrogen or argon. A drop of pure water is applied to the horizontally oriented surface. Adding further water enlarges the droplet surface area, which results in the “upper” contact angle, while the removal of water reduces the droplet diameter in contact with the surface, resulting in the “lower” contact angle. A surface has a hydrophilic character when the “upper” contact angle is less than 50° (<50°) and the “lower” contact angle is less than 20° (<20°).

It is known that organic compounds in air deposit directly onto the surface of titanium and titanium alloys and thus alter the chemistry of the surface. The surface then becomes hydrophobic. Various solutions have already been proposed as to how this problem might be solved and how thereby the osteointegration time of implants might be reduced.

EP 388 576 discloses a metallic implant having a surface roughness of more than 20 μm. On top of this roughness there is a microroughness of not more than 2 μm. It has emerged that organic deposits on the surface have an adverse effect on the osteointegration time.

WO 00/44305 discloses an osteophilic implant having a roughened hydroxylated and hydrophilic surface. At least the hydroxylated and hydrophilic surface is enclosed in a gas- and liquid-tight envelope. The envelope contains an inert atmosphere, for example of nitrogen and/or partly of purified water. One disadvantage with this implant is the relatively complicated packaging process which presupposes a gas- and liquid-tight envelopment with inert atmosphere.

WO 03/030957 discloses an implant having a roughened hydroxylated and hydrophilic surface and being treated in the hydroxylated state with high-energy ultraviolet radiation. One disadvantage of this solution is the additional treatment step which is supposed to be carried out by the surgeon in particular.

U.S. Pat. No. 6,221,111 discloses a bioactive surface coating for a metallic implant. The coating consists of calcium compounds and metal oxides. But the problem of deposits of organic material on the surface of the implant is not solved thereby.

It is an object of the present invention to avoid the disadvantages of the prior art, more particularly to provide an implant, a package and an implant-treating process whereby the impairment of the biologically active surface of the implant due to contaminants is prevented in a simple manner. More particularly, the invention shall not require complicated sterilized packages nor any further costly and inconvenient treating steps.

I have found that these objects are achieved in accordance with the invention by an implant, a package and a process for treating an implant having the features of the independent claims.

The implant of the present invention is in particular a dental implant. The invention can be similarly applied to other implants as well. The implant has an implant body for insertion and incorporation into a bone. The implant is at least partly provided with a protective layer on the layer for incorporation into the bone. This protective layer prevents the deposition of contaminants, in particular organic compounds, on the biologically active surface of the implant. According to the present invention, the layer is configured such that it dissolves on contact with bodily fluid or on contact with the bone. The layer is elaborated such that, after the layer has dissolved, essentially no residues remain on the surface. In addition, the layer is constructed of constituents which, after the layer has dissolved, are generally recognized as safe for the body.

It is particularly preferable for the entire implant surface region which is to come into contact with the bone to be provided with the protective layer. It is also conceivable to provide the complete implant with such a protective layer.

In accordance with another aspect of the invention there is proposed a protective layer composed of a salt. Preferably, the layer consists exclusively of salt. It is conceivable to construct the layer from a single salt or else from a combination of salts.

In general, layers are conceivable which are constructed from additives dissolved in pure water, suitable additives being for example univalent alkali metal cations, such as Na⁺ or K⁺ or a mixture of Na⁺ and K⁺, with corresponding anions in the form of inorganic salts, for example sodium chloride, potassium chloride, sodium chlorate, potassium chlorate, sodium nitrate, potassium nitrate, sodium phosphate, potassium phosphate or a mixture thereof. It is similarly possible to add bivalent cations in the form of water-soluble inorganic salts. Suitable cations are in particular Mg²⁺, Ca²⁺, Sr²⁺ and/or Mn²⁺ in the form of the chlorides or mixtures thereof. Suitable anions further include phosphate and phosphanate anions, which terms each also refer to monoorthophosphate anions and diorthophosphate anions on the one hand and monoorthophosphonate anions and diorthophosphonate anions on the other, in combination with the cations mentioned.

Preferably, the salt comprises cations which occur in human bodily fluid, particular preference being given to cations selected from the group consisting of Na⁺, K⁺, Mg²⁺, Ca²⁺.

In a preferred illustrative embodiment, the layer has a thickness of a few nanometers, in particular 1 to 100 nm, preferably 1 to 10 nm. In principle, it is sufficient for the layer to cover the surface, so that no deposits form thereon. Even a surface layer of ions which is just a few layers of atoms in thickness stops organic compounds depositing directly on the titanium surface. Although the organic compounds are then able to deposit on the salt layer, the surface remains altogether hydrophilic and biologically active as a result of the protection of the ions.

TiOH (titanium hydroxyl) in the outermost atomic layer of the surface is formed by addition of H₂O. Depending on the acid value, the surface is negatively charged (TiO⁻) or positively charged (TiOH₂⁺), and therefore determines which ion is adsorbed in the first atomic layer. The isoelectric point of titanium is in the range pH 6-6.5. Accordingly, anions are adsorbed when the pH is below 6 and cations when the pH is above 6.5.

The biological activity of the surface coated with a protective layer, in particular with ions, can be explained by the fact that, after implantation, water in the bodily fluids is attracted and bound by the layer, in particular ions on the implant surface. This clears the way for the adsorption of diverse ions from the blood, the interaction with biomolecules (proteins, lipids, lipoproteins and peptides) and finally the deposition of bone cells. In the case of rough hydrophobic surfaces, by contrast, air bubbles form in the cavities and hence prevent direct contact of the bodily fluids with the surface. This phenomenon leads to a retardation of the adsorption of biomolecules from the bodily fluids and consequently to a slower osteointegration of the implant.

In a preferred illustrative embodiment, the implant body has a surface having a macroroughness. The macroroughness can typically be obtained by sandblasting with a grain having an average grain size in the range from 0.1 mm to 0.5 mm. Typical such structures are known for example from EP 388 576 and from commercially available implants.

It is particularly preferable for the surface to additionally have microroughness. The production of microroughness on the surface is preferably effected with an inorganic acid or a mixture of inorganic acids, preferably with hydrofluoric acid, hydrochloric acid, sulfuric acid, nitric acid or a mixture thereof.

For example, a treatment can be carried out with an aqueous hydrochloric acid/sulfuric acid mixture having an HCl:H₂SO₄:H₂O ratio of 2:1:1 at >80° C. and for 1 to 10 min. Such a treatment is already known from EP 388 576.

It is similarly preferable for the surface additionally to have nanoroughness. Nanoroughness of the surface is preferably produced using an alkaline solution, in particular an alkali metal hydroxide, preferably using sodium hydroxide or potassium hydroxide.

Preferably, the protective layer consists exclusively of a single salt which has been applied directly to the implant surface after it has been cleaned, in particular freed of organic compounds.

Preferably, the implant consists of titanium or of titanium alloys. But other materials are also conceivable in principle.

The invention shortens the osteointegration time of implants having a layer of water-soluble salt crystals on the chemically clean, rough implant surface. The superior results are particularly observable in the first 6 weeks of the bone wound healing.

A further aspect of the invention provides a package comprising an above-described implant. The implant is kept in a space in the package. The layer forms a preserving layer for preventing deposits on the surface of the implant. There is therefore no need to make the package gas- and/or liquid-tight and, as in the prior art, provided with an inert atmosphere. The manufacturing process is therefore less costly and more convenient.

A further aspect of the present invention provides a process for treating an implant. To this end, the surface of the implant is cleaned, if necessary. Organic deposits in particular are removed in the process. The surface thus cleaned is subsequently provided with a protective layer, in particular with a protective layer of salt.

A particularly simple way of applying the protective layer to the implant is by the implant being dipped into a salt solution and subsequently dried. Typically a 0.01M to 1M solution, in particular a 0.1M NaCl solution can be used. To this end, salts are added to pure water, so that, after drying, the layer deposited on the implant surface consists exclusively of the desired material(s), in particular salts.

In order that no harmful residues are deposited on the surface in the course of drying, drying is particularly carried out with an inert material, for example with nitrogen. Contemplated constituents for the protective layer include in particular the above-described cations and anions.

Immersion of the implant into the solution described and subsequent drying typically produces a layer of a few nanometers, in particular 1 to 100 nm, preferably 1 to 10 nm.

Yet a further aspect of the invention provides the use of a salt layer on the biologically active surface of an implant for protecting at least a portion of the surface of the implant against contaminants.

The invention will now be more particularly described with reference to illustrative embodiments.

### EXAMPLES

The tooth implants are obtained in a conventional manner by turning and milling a cylindrical blank. The surface which comes directly into contact with the bone is provided with a macroroughness by sandblasting it with a grain having an average grain size of 0.1-0.5 mm.

The macroroughened surface is subsequently treated with an aqueous hydrochloric acid/sulfuric acid mixture having a ratio of HCl:H₂SO₄:H₂O of 2:1:1 at a temperature of >80° C. for 1-10 minutes to obtain a defined microroughness on top of the macroroughness.

### Example 1

The implant thus formed is immediately neutralized with a pure 0.15M NaCl solution, removed from the solution and dried with nitrogen. The implant thus provided with a coating is subsequently stored in a package.

### Example 2

The implant thus formed is neutralized in pure water and the roughened surface (macroroughness/micro-roughness) is treated with 3M KOH at a temperature of >60° C. for 10-30 minutes to obtain a defined nanoroughness on top of the micro-/macroroughness. Subsequently, the textured implant is immediately neutralized with a pure 0.15M NaCl solution, removed from the solution and dried with nitrogen. The implant thus provided with a salt layer is subsequently stored in a package.

### Example 3

The implant thus formed is neutralized in pure water and air dried at 80-110° C. Thereafter, the surface is cleaned with a UV/ozone treatment and immediately dipped into a pure 0.15M NaCl solution before it is removed from the solution and dried with nitrogen. In this second illustrative embodiment, any contaminants which have become redeposited after the acid treatment can be removed. The implant thus protected is placed in a package and kept therein.

### Example 4

The implant thus formed is neutralized in pure water and air dried at 80-110° C. Thereafter, the surface is cleaned with a plasma treatment and immediately dipped into a pure 0.15M NaCl solution before it is removed from the solution and dried with nitrogen.

The coating of the surface with ions can be analyzed by x-ray photoelectron spectroscopy (XPS) and Auger electron spectroscopy (AES). A good coating with ions is, as explained above, just a few nm in thickness and comprises 1-50 atom percent of positively and negatively charged ions.

Results of XPS measurement on use of:

### 0.01M NaCl: 5% Na⁺, 2% Cl⁻

### 0.1M NaCl: 20% Na⁺, 12% Cl⁻

### 1M NaCl: 40% Na⁺, 25% Cl⁻

How firmly the implant is integrated in the bone can be determined mechanically, namely by measuring the force, whether as tension, pressure, shearing or torque, needed to pull or twist the implant attached to the bone out of its attachment. Measurements have shown that titanium implants having a smooth surface texture become only insufficiently well attached in the bone, while implants having a roughened surface produce a markedly improved bone-implant bond in terms of tensile strength. It has emerged that implants having a coating of ions give superior osteointegration than implants without salt coating.

A further way of ascertaining improved osteointegration is by measuring the bone implant. To this end, histological sections of the implant in the bone are systematically analyzed under the optical microscope. It has again emerged that implants having a coating of ions give superior osteointegration than implants without salt coating.

1-20. (canceled)

21. An implant, comprising an implant body for insertion into a bone, wherein at least the implant body is at least partly provided with a protective layer which dissolves on contact with body fluid and/or the bone.

22. The implant of claim 21 comprising an implant body for insertion into a bone, wherein at least the implant body is at least partly provided with a protective layer consisting of salt.

23. The implant according to claim 22 wherein the salt consists of cations which occur in human bodily fluid, in particular of cations selected from the group consisting of Na+, K+, Mg2+, Ca2+.

24. The implant according to claim 23 wherein the anion of the salt is Cl- or phosphate.

25. The implant according to claim 24 wherein the layer has a density of a few nanometers.

26. The implant according to claim 25 wherein the layer has a density of 1 to 100 nm.

27. The implant according to claim 26 wherein the layer has a density of 1 to 10 nm.

28. The implant according to claim 21, wherein the implant body has a surface having a macroroughness.

29. The implant according to claim 28, wherein the macroroughness is obtainable by sandblasting with a grain having an average grain size in the range from 0.1 mm to 0.5 mm.

30. The implant according to claim 21, wherein the implant body has a surface having a microroughness.

31. The implant according to claim 30, wherein the microroughness is obtainable via acid treatment.

32. The implant according to claim 31, wherein the microroughness is obtainable via treatment with an aqueous hydrochloric acid/sulfuric acid mixture having an HCl:H2SO4:H2O ratio of 2:1:1 at >80° Celsius for 1 to 10 min.

33. The implant according to claim 21, wherein a surface has a nanoroughness.

34. The implant according to claim 21, wherein the nanoroughness is obtainable by alkaline etching.

35. The implant according to claim 34, wherein the nanoroughness is obtainable via treatment with a solution of an alkali metal hydroxide.

36. The implant according to claim 35, wherein the nanoroughness is obtainable with sodium hydroxide or potassium hydroxide.

37. The implant according to claim 36, wherein the nanoroughness is obtainable with sodium hydroxide or potassium hydroxide at a concentration in the range of 1-5M, at >55° C., in for 10-30 minutes.

38. The implant to claim 22, wherein the layer consists exclusively of a single salt applied directly to a precleaned surface.

39. The implant according to claim 21, wherein the implant body consists of titanium or a titanium alloy.

40. The implant according to claim 21 wherein the implant is a dental implant.

41. A package comprising an implant according to claim 21, wherein the implant is held in a space in the package and wherein the protective layer, in particular the salt layer, forms a layer for preventing deposits on the surface of the implant.

42. A process for treating an implant, wherein the surface of the implant is at least partly provided with a protective layer which dissolves on contact with bodily fluid, and wherein said protective layer is a layer of salt.

43. The process according to claim 42 wherein the surface of the implant is cleaned, in particular freed of organic deposits, before application of the protective layer.

44. The process according to claim 42, wherein the layer is applied by dipping the implant into a salt solution and by subsequent drying.

45. The process according to claim 42, wherein the implant is dipped into a 0.01M-1M NaCl solution.

46. The process according to claim 42, wherein the surface is dried after removal from the solution with an inert material.

47. The process according to claim 44, wherein the solution comprises cations which occur in human body fluid.

48. The process according to claim 44, wherein the anion in the solution is Cl- or phosphate.

49. The process according to claim 42 wherein the protective layer is applied in a thickness of a few nanometers.
